(19) United States
(12) Patent Application Publication
(10) Pub. No.: US 2024/0270696 A1
(43) Pub. Date: Aug. 15, 2024

(54) FULL CONTINUOUS SYNTHESIS DEVICE AND METHOD FOR METRONIDAZOLE

(71) Applicant: Fudan University, Shanghai (CN)

(72) Inventors: Fener CHEN, Shanghai (CN); Minjie LIU, Shanghai (CN); Li WAN, Shanghai (CN); Yajiao ZHANG, Shanghai (CN); Dang CHENG, Shanghai (CN)

(21) Appl. No.: 18/625,925

(22) Filed: Apr. 3, 2024

(30) Foreign Application Priority Data
Nov. 1, 2023 (CN) 202311442699.2

(51) Int. Cl. C07D 233/94; B01J 19/00

(52) U.S. Cl. CPC C07D 233/94; B01J 19/006; B01J 19/0066; B01J 19/0093; B01J 2219/00033; B01J 2219/00768; B01J 2219/00783; B01J 2219/00864; B01J 2219/00867; B01J 2219/00889

(57) ABSTRACT

A full continuous synthesis method of metronidazole is provided. An aqueous glyoxal solution, an aqueous acetaldehyde solution and aqueous ammonia are mixed and reacted to produce a 2-methylimidazole-containing reaction mixture, which is mixed with a nitric acid solution and then reacted in the presence of concentrated sulfuric acid to obtain a 2-methyl-5-nitroimidazole-containing reaction mixture. The 2-methyl-5-nitroimidazole-containing reaction mixture is divided by a splitter, such that one part is used to replace concentrated sulfuric acid, and the other part is mixed with formic acid, and undergoes a ring-opening reaction with ethylene oxide to obtain a metronidazole solution. The metronidazole solution is adjusted to pH 2-6 and filtered to obtain a filtrate, which is adjusted to pH 8-14 and filtered to obtain a crude product. The crude product is subjected to decoloring, crystallization, filtration and drying to obtain pure metronidazole with a purity greater than 99.9%.

### CROSS-REFERENCE TO RELATED APPLICATIONS

This application claims the benefit of priority from Chinese Patent Application No. 202311442699.2, filed on Nov. 1, 2023. The content of the aforementioned application, including any intervening amendments thereto, is incorporated herein by reference in its entirety.

### TECHNICAL FIELD

This application relates to pharmaceutical chemicals, and more particularly to a full continuous synthesis device and method for metronidazole.

### BACKGROUND

Metronidazole (C₆H₉N₃O₃ with CAS number of 443-48-1), also called 1-(2-Hydroxyethyl)-2-methyl-5-nitroimidazole, is a white or yellowish crystal or crystalline powder. Metronidazole is a nitroimidazole antiprotozoal and anti-anaerobic drug mainly used in clinical prevention and treatment of anaerobic infections, and has a large global clinical demand. Metronidazole has been approved by the World Health Organization as an essential drug against anaerobic infections in 1978.

At present, the production of metronidazole generally adopts a batch reactor, which has long reaction time, high energy consumption, low conversion of 2-methyl-5-nitroimidazole (main raw material), serious by-reaction of ethylene oxide and large production of waste acid and salt. Chinese Patent Publication No. 110669011A developed a microtubular reaction process for synthesizing metronidazole from 2-methyl-5-nitroimidazole. The raw materials are mixed in the mixing zone of a commercially-available pre-packed coiled reactor, and then enter the rection zone with an inner diameter of 4-20 mm for hydroxyethylation, which avoids volatilization of ethylene oxide with a low boiling point and improves utilization efficiency of ethylene oxide. However, it is required to cool ethylene oxide into a low-temperature liquid in advance, which increases the energy consumption. Moreover, due to the low boiling point, the vapor pressure of ethylene oxide varies significantly under different room temperature conditions, which renders the measuring pump less accurate, thereby affecting the conversion rate and selectivity of the reaction. Chinese Patent Publication No. 111574459A developed a preparation method of metronidazole from 2-methyl-5-nitroimidazole, in which the ethylene oxide is added batchwise to a kettle reactor, such that the volatilization of ethylene oxide is alleviated, and the utilization efficiency of ethylene oxide is increased. However, the batchwise addition of ethylene oxide is time-consuming, and has complex operation and low production efficiency.

### SUMMARY

In view of the problems of the prior art, this application provides a full continuous synthesis device and method for metronidazole, which has high production efficiency, excellent product quality and less emission of three wastes (waste water, waste gas and industrial residue).

The present disclosure provides a full continuous synthesis method for metronidazole using 2-methylimidazole as a starting material, in which a micromixer, a microreactor, a self-designed solvent switching system and a self-designed ethylene oxide conveying system are integrated to synthesize high-purity metronidazole product through three-step reactions and continuous operation, and realize the recovery of sulfuric acid and formic acid, which enables the accurate and continuous feeding of ethylene oxide, and significantly mitigates the emission of the three wastes. Based on the fully-continuous synthesis, the production efficiency and control accuracy are greatly improved, thereby ensuring the stability of product quality.

The present disclosure provides a full continuous synthesis method for metronidazole using a full continuous synthesis device, the full continuous synthesis device comprising a first mixer, a first microreactor, a first solvent switching system, a second mixer, a second microreactor, an ethylene oxide conveying system, a third microreactor and a second solvent switching system communicated in sequence; and the full continuous synthesis method comprising:
(a) mixing an aqueous glyoxal solution and an aqueous acetaldehyde solution to obtain a first reagent, and mixing the first reagent with aqueous ammonia in the first mixer followed by reaction in the first microreactor to produce a 2-methylimidazole-containing reaction mixture; removing unreacted glyoxal, acetaldehyde, and ammonia and water from the 2-methylimidazole-containing reaction mixture in the first solvent switching system; and mixing a 2-methylimidazole-containing outflow from the first solvent switching system with a nitric acid solution to obtain a second reagent;
(b) mixing the second reagent with concentrated sulfuric acid in the second mixer followed by reaction in the second microreactor to produce a 2-methyl-5-nitroimidazole-containing reaction mixture; removing unreacted nitric acid from the 2-methyl-5-nitroimidazole-containing reaction mixture in a negative-pressure tank to obtain a 2-methyl-5-nitroimidazole sulfuric acid solution; and delivering the 2-methyl-5-nitroimidazole sulfuric acid solution to a splitter such that a first part of the 2-methyl-5-nitroimidazole sulfuric acid solution is used as a third reagent to participate in a next step, and a second part of the 2-methyl-5-nitroimidazole sulfuric acid solution is used to replace concentrated sulfuric acid to react with 2-methylimidazole to improve a concentration of 2-methyl-5-nitroimidazole;
(c) mixing the third reagent with formic acid followed by mixing with ethylene oxide quantitatively fed by the ethylene oxide conveying system to obtain a reaction mixture; subjecting the reaction mixture to a ring-opening reaction in the third microreactor to obtain a crude metronidazole solution; and conveying the crude metronidazole solution to the second solvent switching system to remove and recover formic acid; and
(d) adjusting a residual metronidazole solution to pH 2-6 in a multi-function stirred tank of the second solvent switching system followed by filtration to obtain a filtrate and recover 2-methyl-5-nitroimidazole; adjusting the filtrate to pH 8-14 followed by filtration to obtain a crude metronidazole product; and subjecting the crude metronidazole product to decoloring, crystallization, filtration and drying to obtain pure metronidazole with a purity greater than 99.9%.

In an embodiment, the first mixer and the second mixer are specially designed and manufactured and each have a plate-type homocentric-square channel structure with a width of 100 μm-20 mm, a length of 1 m-2000 m and an applicable flux of 1 mL/min-3000 mL/min.

In an embodiment, the first microreactor, the second microreactor and the third microreactor are each independently have a plate-type X-shaped channel structure with a fluid channel size of 100μm-20 mm or a tubular baffle filled channel structure with a fluid channel size of 300μm-50 mm (referring to FIGS. 5a-5b).

In an embodiment, the first solvent switching system and the second solvent switching system are specially designed and manufactured, and is provided in two schemes. Referring to FIG. 2, the first solvent switching system and the second solvent switching system each comprises the multi-function stirred tank, a vacuum system, a cooling system and a heating circulation system. The vacuum system, the cooling system and the heating circulation system are connected to the multi-function stirred tank. The cooling system is a graham condenser. The vacuum system is a vacuum pump. The heating circulation system is a heating-cooling integrated machine or a circulating heating oil bath. The vacuum system, the cooling system and the heating circulation system are connected to the multi-function stirred tank through a pipe with a flange. A tank body of the multi-function stirred tank adopts a jacketed heat exchange structure which is configured that a heat exchange fluid enters the tank body through a lower port and leaves from the tank body from an upper port. An S-shaped baffle is provided in a heat exchange channel of the jacketed heat exchange structure. A top of the multi-function stirred tank is provided with a material inlet (for the first solvent switching system, the material inlet is configured for feeding of the 2-methylimidazole-containing reaction mixture, and for the second solvent switching system, the material inlet is configured for feeding of the crude metronidazole solution). A lower end of the first material inlet in configured to extend into a position, which is away from a bottom of the tank body at a distance of ¼ height of the tank body. A top of the tank body is provided with a first outlet connected with a bottom of the graham condenser. The bottom of the graham condenser is provided with a second outlet connected with a liquid storage tank for collecting condensate. A lower portion of the graham condenser is provided with a coolant inlet, and an upper portion of the graham condenser is provided with a coolant outlet; the coolant inlet is configured to allow a coolant to enter a coiled tube in the graham condenser, and the coolant outlet is configured to allow the coolant to leave the coil tube. A top of the graham condenser is in pipeline connection with an adjusting valve and the vacuum pump. The vacuum pump is configured to provide negative pressure. The adjusting valve is configured to adjust negative pressures in the multi-function stirred tank and the graham condenser, so as to control a gasification velocity of low-boiling-point compounds and avoid incomplete condensation in the graham condenser. The heating circulation system is connected with the multi-function stirred tank through the pipe with the flange. A heat flow is pumped by a circulating pump. After removing the low-boiling-point compounds, adding the nitric acid solution, that is, the low-boiling-point compounds, such as water, are removed from a reaction liquid in the multi-function stirred tank, and then a solvent is switched to the nitric acid solution. In order to realize continuous operation, two sets of the multi-function stirred tank are used in parallel and cross to achieve a continuous preparation of the second reagent and a continuous application of a next nitrification reaction.

In an embodiment, referring to FIG. 3 the first solvent switching system and the second solvent switching system each have a zigzag baffle-filled vertical channel with a nitrogen purging function (shown in FIG. 3). The zigzag baffle-filled vertical channel of the first solvent switching system is configured to concentrate the 2-methylimidazole-containing reaction mixture. Six groups of zigzag baffles are provided in the zigzag baffle-filled vertical channel. An outer wall of the zigzag baffle-filled vertical channel is provided with a heat exchange jacket, a bottom of the zigzag baffle-filled vertical channel is provided with a nitrogen inlet, and a top of the zigzag baffle-filled vertical channel is provided with a nitrogen outlet. The six groups of zigzag baffles are configured to increase a liquid dispersion area and accelerate gasification of the low-boiling-point compounds. The heat exchange jacket is configured to ensure temperature of liquid in the vertical channel and the prevent re-condensation after gasification. The 2-methylimidazole-containing reaction mixture is preheated before entering the first solvent switching system, and the crude metronidazole solution is preheated before entering the second solvent switching system. A second material inlet is configured to extend from the top of the vertical channel to a position which is away from the top of the vertical channel at a distance of 1/10 height of the vertical channel. The nitrogen inlet is provided at the bottom of the vertical channel. The nitrogen is fully contacted with the liquid on the six groups of zigzag baffles to accelerate gasification of the low-boiling-point compounds, and the low-boiling-point compounds are quickly brought out of the vertical channel with a flow of nitrogen, which avoids re-liquefaction of the low-boiling-point compounds, realizes quick evaporation of the low-boiling-point compounds below their boiling points, and is suitable for solvent switching of unstable compounds which are temperature-sensitive. The nitrogen outlet is connected with the graham condenser in the multi-function stirred tank to recover low-boiling-point compounds.

In an embodiment, the splitter is an adjustable liquid flowmeter connected with a valve. The splitter is configured such that a part of the 2-methyl-5-nitroimidazole sulfuric acid solution obtained after a first nitrification reaction in the second microreactor is used to replace the concentrated sulfuric acid to participate in a second nitrification reaction in the second microreactor to obtain a primarily-concentrated 2-methyl-5-nitroimidazole sulfuric acid solution. The splitter is opened to deliver the primarily-concentrated 2-methyl-5-nitroimidazole sulfuric acid solution to participate a next step reaction, or the primarily-concentrated 2-methyl-5-nitroimidazole sulfuric acid solution is used to participate in a third nitrification reaction in the second microreactor to obtain a secondarily-concentrated 2-methyl-5-nitroimidazole sulfuric acid solution, and so forth until a saturated 2-methyl-5-nitroimidazole sulfuric acid solution is obtained which is used in a next step through the splitter, so as to save a consumption of concentrated sulfuric acid in the nitration reaction and realize that one sulfuric acid is used multiple times to nitrate multiple 2-methylimidazole.

In an embodiment, the ethylene oxide conveying system is specially designed and manufactured. The ethylene oxide conveying system includes a buffer tank provided with an ethylene oxide inlet pipe and an ethylene oxide outlet pipe. The ethylene oxide inlet pipe is arranged in the buffer tank at ½ height of the buffer tank, and the ethylene oxide outlet pipe is configured to extend from a bottom of the buffer tank to a position inside the buffer tank which is away from the bottom of the buffer tank at a distance of ⅕-¼ height of the buffer tank. The buffer tank is pressurized to 5 bar-10 bar with nitrogen after filled with ethylene oxide. The ethylene oxide conveying system further includes an injection pump; and the injection pump is configured to quantitatively feed the ethylene oxide to be mixed with a 2-methyl-5-nitroimidazole sulfuric acid-formic acid mixture to prepare metronidazole.

In an embodiment, in step (d), a pH value of the residual metronidazole solution is monitored by an on-line pH meter, and is adjusted with aqueous ammonia or liquid ammonia; the residual metronidazole solution is pumped to an on-line filter through a plunger pump to obtain a first filtrate; and the first filtrate is returned to a first pH-adjusting tank until a pH value of the first filtrate is kept within 2-6.

The first filtrate with the pH value of 2-6 is adjusted with aqueous ammonia or liquid ammonia in a second pH-adjusting tank pumped to the on-line filter through the plunger pump to obtain a second filtrate, and the second filtrate is returned to the second pH-adjusting tank until a pH value of the second filtrate is kept within 8-14. A filter cake of the on-line filter is continuously scraped by using a rotatory scraper to obtain the crude metronidazole. The crude metronidazole is decolorized and crystallized to obtain pure metronidazole with the purity greater than 99.9%.

Compared to the prior art, this application has the following beneficial effects.

In the present disclosure, the efficient continuous synthesis of metronidazole is realized through reasonable combination of a series of micromixers and microreactor according to synthesis process of metronidazole. Moreover, the continuous synthesis of metronidazole is realized through the solvent switching system designed herein, which greatly improves the production efficiency and realizes the recovery and utilization of excess nitric acid and formic acid. By means of the splitter, the sulfuric acid can be repeatedly recycled, such that the sulfuric acid consumption can be reduced by more than 80%, thereby efficiently reducing the production of waste acid and waste salt and reducing the production cost. In addition, the ethylene oxide conveying system designed herein can effectively ensure the stable and accurate feeding of ethylene oxide to the pressurized microchannel reactor.

### BRIEF DESCRIPTION OF THE DRAWINGS

FIG. 1 is a flow chart of a full continuous synthesis method for metronidazole according to an embodiment of the present disclosure.

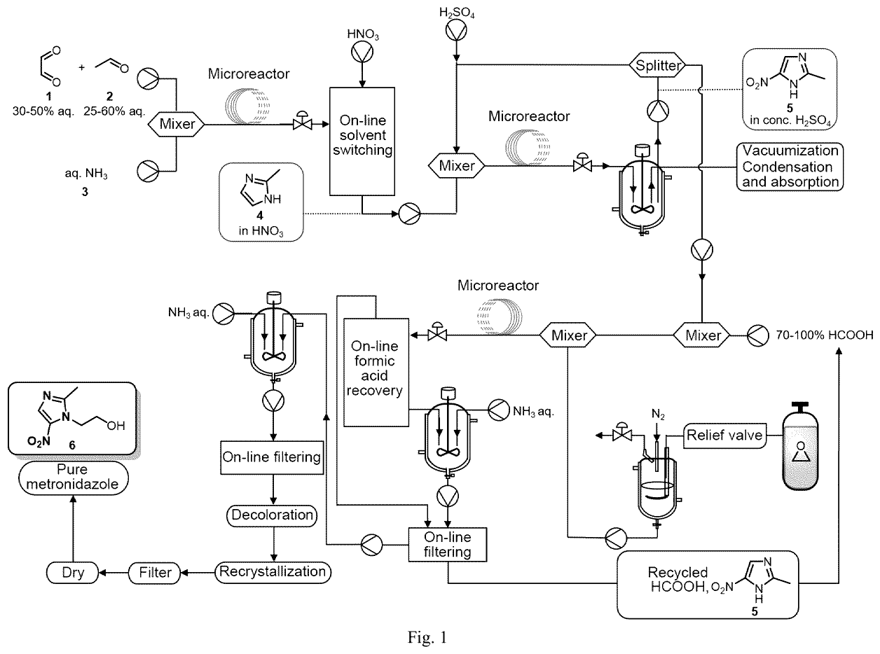

FIG. 2 is a structural diagram of a multi-function stirred tank connected with a vacuum system, a cooling system and a heating circulation system according to an embodiment of the present disclosure.

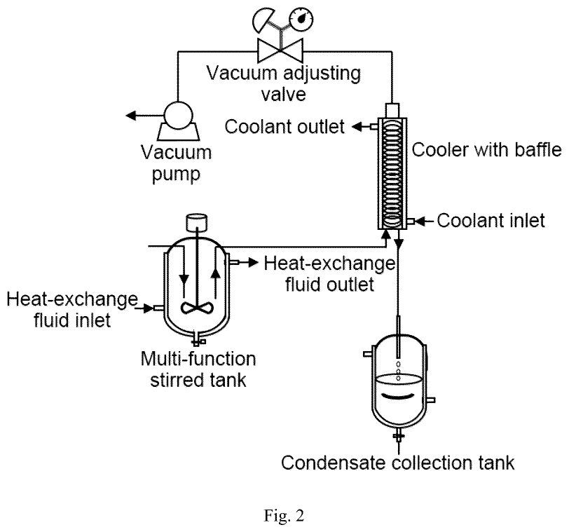

FIG. 3 schematically shows a zigzag baffle-filled vertical channel with nitrogen purging function according to an embodiment of the present disclosure.

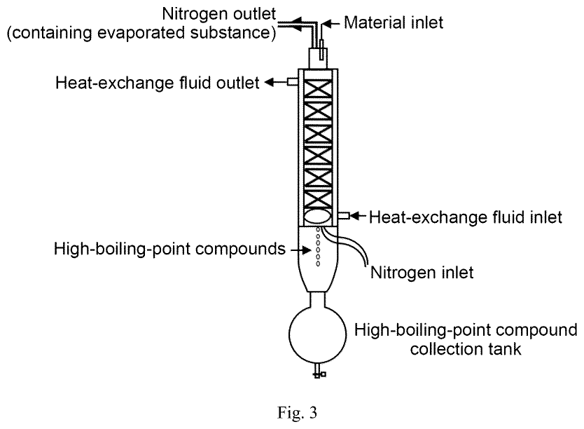

FIG. 4 schematically shows a micromixer with a homocentric-square structure according to an embodiment of the present disclosure.

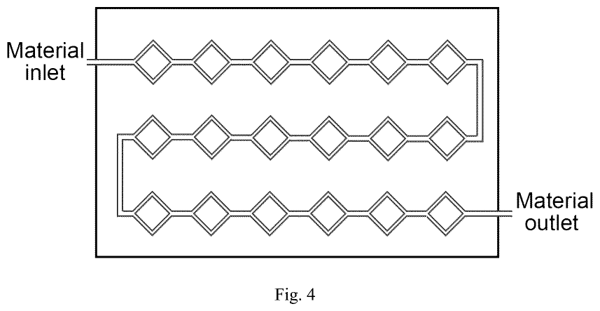

FIG. 5a schematically shows an X-shaped channel microreactor according to an embodiment of the present disclosure.

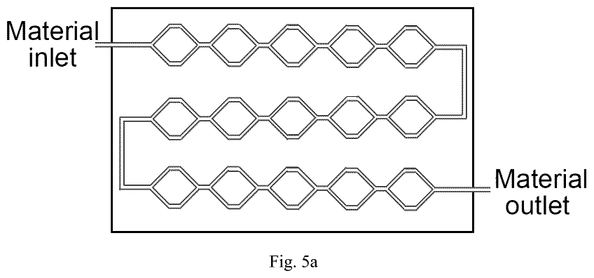

FIG. 5b schematically shows a tubular baffle-filled channel microreactor according to an embodiment of the present disclosure.

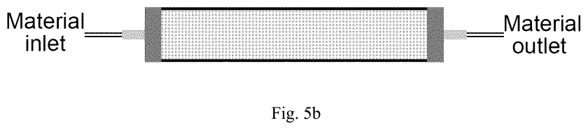

### DETAILED DESCRIPTION OF EMBODIMENTS

In order to illustrate the technical solutions, structure characteristics, objectives and effects of the present disclosure in detail, the present disclosure will be described below with reference to the accompanying drawings and embodiments. The embodiments described below are illustrative, and are not intended to limit the scope of this application.

In order to make purposes, technical solutions and beneficial effects of the present disclosure clearer, the present disclosure will be clearly and completely described below with reference to the accompanying drawings and embodiments.

### Example 1

An aqueous glyoxal solution was mixed with an aqueous acetaldehyde water solution in a molar ratio of 1:1 to obtain a reagent A. The reagent A and aqueous ammonia (1.2 equiv., reagent B) were pumped to a homocentric-square mixer through a plunger pump, fully mixed and reacted in an X-shaped microreactor to produce a 2-methylimidazole-containing reaction mixture. The 2-methylimidazole-containing reaction mixture is treated under reduced pressure in a multi-function stirred tank connected with a vacuum system, a cooling system and a heating circulation system to remove unreacted aldehyde, ammonia and water. The resultant 2-methylimidazole was mixed with a 70% nitric acid solution (1.2 equiv.) to obtain a reagent C, which was mixed with concentrated sulfuric acid in another homocentric-square mixer and then reacted in another X-shaped microreactor to produce a 2-methyl-5-nitroimidazole-containing reaction mixture. The 2-methyl-5-nitroimidazole-containing reaction mixture was conveyed to a negative-pressure tank to remove the unreacted nitric acid, so as to obtain a 2-methyl-5-nitroimidazole sulfuric acid solution. The 2-methyl-5-nitroimidazole sulfuric acid solution was used to replace the concentrated sulfuric acid to participate in the nitration of the 2-methylimidazole to produce a primarily-concentrated 2-methyl-5-nitroimidazole sulfuric acid solution, and such operation was repeated again to obtain a secondarily-concentrated 2-methyl-5-nitroimidazole sulfuric acid solution as reagent D. In this way, one batch of sulfuric acid was used to participate in three batches of 2-methylimidazole nitration, reducing the consumption of sulfuric acid. The reagent D was mixed with formic acid and then mixed with ethylene oxide (1.1 equiv.) quantitatively output from an ethylene oxide conveying system, and subjected to a ring-opening reaction in another X-shaped microreactor to obtain a final product metronidazole. The metronidazole-containing reaction liquid entered a spiral channel with a zigzag baffle to remove formic acid by condensation and recovery. The residual reaction liquid was adjusted to pH 2-4, and filtered to recover 2-methyl-5-nitroimidazole. The filtrate was further adjusted to pH 9-12, and filtered to obtain crude metronidazole. The crude metronidazole is decolored, crystallized, filtered and dried to obtain pure metronidazole with a purity greater than 99.9%. Compared to the conventional one-through synthesis, the consumption of sulfuric acid of this embodiment was reduced by 65%; the recovery rate of formic acid was 95%; a one-through yield of metronidazole was 80%; and the yield of 2-methyl-5-nitroimidazole reached 96% after repeated recycling.

### Example 2

An aqueous glyoxal solution was mixed with an aqueous acetaldehyde water solution in a molar ratio of 1:1 to obtain a reagent A. The reagent A and aqueous ammonia (1.2 equiv., reagent B) were pumped to a homocentric-square mixer through a plunger pump, fully mixed and reacted in an X-shaped microreactor to produce a 2-methylimidazole-containing reaction mixture. The 2-methylimidazole-containing reaction mixture is treated under reduced pressure in a zigzag baffle-filled vertical channel to be concentrated and remove low-boiling-point compounds and water. The resultant 2-methylimidazole was mixed with a 70% nitric acid solution (1.2 equiv.) to obtain a reagent C, which was mixed with concentrated sulfuric acid in another homocentric-square mixer and then reacted in another X-shaped microreactor to produce a 2-methyl-5-nitroimidazole-containing reaction mixture. The 2-methyl-5-nitroimidazole-containing reaction mixture was conveyed to a negative-pressure tank to remove the unreacted nitric acid, so as to obtain a 2-methyl-5-nitroimidazole sulfuric acid solution. The 2-methyl-5-nitroimidazole sulfuric acid solution was used to replace the concentrated sulfuric acid to participate in the nitration of the 2-methylimidazole to produce a concentrated 2-methyl-5-nitroimidazole sulfuric acid solution as reagent D. In this way, one batch of sulfuric acid was used to participate in two batches of 2-methylimidazole nitration, reducing the consumption of sulfuric acid. The reagent D was mixed with formic acid and then mixed with ethylene oxide (1.1 equiv.) quantitatively output from an ethylene oxide conveying system, and subjected to a ring-opening reaction in another X-shaped microreactor to obtain a final product metronidazole. The metronidazole-containing reaction liquid entered a spiral channel with a zigzag baffle to remove formic acid by condensation and recovery. The residual reaction liquid was adjusted to pH 2-4, and filtered to recover 2-methyl-5-nitroimidazole. The filtrate was further adjusted to pH 9-12, and filtered to obtain crude metronidazole. The crude metronidazole is decolored, crystallized, filtered and dried to obtain pure metronidazole with a purity greater than 99.9%. Compared to the conventional one-through synthesis, the consumption of sulfuric acid of this embodiment was reduced by 45%; the recovery rate of formic acid was 95%; a one-through yield of metronidazole was 75%; and the yield of 2-methyl-5-nitroimidazole reached 93% after repeated recycling.

### Example 3

An aqueous glyoxal solution was mixed with an aqueous acetaldehyde water solution in a molar ratio of 1:1 to obtain a reagent A. The reagent A and aqueous ammonia (1.2 equiv., reagent B) were pumped to a homocentric-square mixer through a plunger pump, fully mixed and reacted in an X-shaped microreactor to produce a 2-methylimidazole-containing reaction mixture. The 2-methylimidazole-containing reaction mixture is treated under nitrogen purging and heating condition in a zigzag baffle-filled vertical channel to be concentrated and remove low-boiling-point compounds and water. The resultant 2-methylimidazole was mixed with a 70% nitric acid solution (1.2 equiv.) to obtain a reagent C, which was mixed with concentrated sulfuric acid in another homocentric-square mixer and then reacted in another X-shaped microreactor to produce a 2-methyl-5-nitroimidazole-containing reaction mixture. The 2-methyl-5-nitroimidazole-containing reaction mixture was conveyed to a negative-pressure tank to remove the unreacted nitric acid, so as to obtain a 2-methyl-5-nitroimidazole sulfuric acid solution. The 2-methyl-5-nitroimidazole sulfuric acid solution was used to replace the concentrated sulfuric acid to participate in the nitration of the 2-methylimidazole to produce a concentrated 2-methyl-5-nitroimidazole sulfuric acid solution as reagent D. In this way, one batch of sulfuric acid was used to participate in two batches of 2-methylimidazole nitration, reducing the consumption of sulfuric acid. The reagent D was mixed with formic acid and then mixed with ethylene oxide (1.1 equiv.) quantitatively output from an ethylene oxide conveying system, and subjected to a ring-opening reaction in another X-shaped microreactor to obtain a final product metronidazole. The metronidazole-containing reaction liquid entered a spiral channel with a zigzag baffle to remove formic acid using nitrogen purging and preheating by condensation and recovery. The residual reaction liquid was adjusted to pH 2-4, and filtered to recover 2-methyl-5-nitroimidazole. The filtrate was further adjusted to pH 9-12, and filtered to obtain crude metronidazole. The crude metronidazole is decolored, crystallized, filtered and dried to obtain pure metronidazole with a purity greater than 99.9%. Compared to the conventional one-through synthesis, the consumption of sulfuric acid of this embodiment was reduced by 45%; the recovery rate of formic acid was 98%; a one-through yield of metronidazole was 82%; and the yield of 2-methyl-5-nitroimidazole reached 95% after repeated recycling.

It should be noted that the embodiments described above are only to illustrate this application rather than limiting the scope of this application. Therefore, any other changes and modifications made by those skilled in the art without departing from the spirit of the application shall fall within the scope of this application defined by the appended claims.

What is claimed is:

1. A full continuous synthesis method for metronidazole using a full continuous synthesis device, the full continuous synthesis device comprising a first mixer, a first microreactor, a first solvent switching system, a second mixer, a second microreactor, an ethylene oxide conveying system, a third microreactor and a second solvent switching system communicated in sequence; and the full continuous synthesis method comprising:
(a) mixing an aqueous glyoxal solution and an aqueous acetaldehyde solution to obtain a first reagent, and mixing the first reagent with aqueous ammonia in the first mixer followed by reaction in the first microreactor to produce a 2-methylimidazole-containing reaction mixture; removing unreacted glyoxal, acetaldehyde, and ammonia and water from the 2-methylimidazole-containing reaction mixture in the first solvent switching system; and mixing a 2-methylimidazole-containing outflow from the first solvent switching system with a nitric acid solution to obtain a second reagent;
(b) mixing the second reagent with concentrated sulfuric acid in the second mixer followed by reaction in the second microreactor to produce a 2-methyl-5-nitroimidazole-containing reaction mixture; removing unreacted nitric acid from the 2-methyl-5-nitroimidazole-containing reaction mixture in a negative-pressure tank to obtain a 2-methyl-5-nitroimidazole sulfuric acid solution; and delivering the 2-methyl-5-nitroimidazole sulfuric acid solution to a splitter such that a first part of the 2-methyl-5-nitroimidazole sulfuric acid solution is used as a third reagent to participate in a next step, and a second part of the 2-methyl-5-nitroimidazole sulfuric acid solution is used to replace concentrated sulfuric acid to react with 2-methylimidazole to improve a concentration of 2-methyl-5-nitroimidazole;
(c) mixing the third reagent with formic acid followed by mixing with ethylene oxide quantitatively fed by the ethylene oxide conveying system to obtain a reaction mixture; subjecting the reaction mixture to a ring-opening reaction in the third microreactor to obtain a crude metronidazole solution; and conveying the crude metronidazole solution to the second solvent switching system to remove and recover formic acid; and
(d) adjusting a residual metronidazole solution to pH 2-6 in a multi-function stirred tank of the second solvent switching system followed by filtration to obtain a filtrate and recover 2-methyl-5-nitroimidazole; adjusting the filtrate to pH 8-14 followed by filtration to obtain a crude metronidazole product; and subjecting the crude metronidazole product to decoloring, crystallization, filtration and drying to obtain pure metronidazole with a purity greater than 99.9%.

2. The full continuous synthesis method of claim 1, wherein the first mixer and the second mixer each have a plate-type homocentric-square channel structure with a width of 100 μm-20 mm, a length of 1 m-2000 m and an applicable flux of 1 mL/min-3000 mL/min.

3. The full continuous synthesis method of claim 1, wherein the first microreactor, the second microreactor and the third microreactor are each independently have a plate-type X-shaped channel structure with a fluid channel size of 100μm-20 mm or a tubular baffle filled channel structure with a fluid channel size of 300μm-50 mm.

4. The full continuous synthesis method of claim 1, wherein the first solvent switching system and the second solvent switching system each comprises the multi-function stirred tank, a vacuum system, a cooling system and a heating circulation system; the vacuum system, the cooling system and the heating circulation system are connected to the multi-function stirred tank; the cooling system is a graham condenser; the vacuum system is a vacuum pump; the heating circulation system is a heating-cooling integrated machine or a circulating heating oil bath; the vacuum system, the cooling system and the heating circulation system are connected to the multi-function stirred tank through a pipe with a flange; a tank body of the multi-function stirred tank adopts a jacketed heat exchange structure which is configured such that a heat exchange fluid enters the tank body through a lower port and leaves from the tank body from an upper port; an S-shaped baffle is provided in a heat exchange channel of the jacketed heat exchange structure; a top of the multi-function stirred tank is provided with a material inlet; for the first solvent switching system, the material inlet is configured for feeding of the 2-methylimidazole-containing reaction mixture, and for the second solvent switching system, the material inlet is configured for feeding of the crude metronidazole solution; a lower end of the material inlet is configured to extend into a position, which is away from a bottom of the tank body at a distance of ¼ height of the tank body; a top of the tank body is provided with a first outlet connected with a bottom of the graham condenser; the bottom of the graham condenser is provided with a second outlet in pipeline connection with a liquid storage tank for collecting a condensate; a lower portion of the graham condenser is provided with a coolant inlet, and an upper portion of the graham condenser is provided with a coolant outlet; the coolant inlet is configured to allow a coolant to enter a coiled tube in the graham condenser, and the coolant outlet is configured to allow the coolant to leave the coil tube; a top of the graham condenser is in pipeline connection with an adjusting valve and the vacuum pump; the vacuum pump is configured to provide negative pressure; the adjusting valve is configured to adjust negative pressures in the multi-function stirred tank and the graham condenser, so as to control a gasification velocity of volatile compounds and avoid incomplete condensation in the graham condenser; the heating circulation system is connected with the multi-function stirred tank through the pipe with the flange; a heat flow is pumped by a circulating pump.

5. The full continuous synthesis method of claim 1, wherein the first solvent switching system and the second solvent switching system each have a zigzag baffle-filled vertical channel with a nitrogen purging function; the zigzag baffle-filled vertical channel of the first solvent switching system is configured to concentrate the 2-methylimidazole-containing reaction mixture; six groups of zigzag baffles are provided in the zigzag baffle-filled vertical channel; an outer wall of the zigzag baffle-filled vertical channel is provided with a heat exchange jacket, a bottom of the zigzag baffle-filled vertical channel is provided with a nitrogen inlet, and a top of the zigzag baffle-filled vertical channel is provided with a nitrogen outlet; the six groups of zigzag baffles are configured to increase a liquid dispersion area and accelerate gasification of the low-boiling-point compounds; the heat exchange jacket is configured to ensure temperature of liquid in the vertical channel and prevent re-condensation after gasification; the 2-methylimidazole-containing reaction mixture is preheated before entering the first solvent switching system, and the crude metronidazole solution is preheated before entering the second solvent switching system; a material inlet is configured to extend from the top of the vertical channel to a position which is away from the top of the vertical channel at a distance of 1/10 height of the vertical channel; the nitrogen inlet is provided at the bottom of the vertical channel; the nitrogen outlet is connected with a graham condenser in the multi-function stirred tank to recover low-boiling-point compounds.

6. The full continuous synthesis method of claim 1, wherein the splitter is an adjustable liquid flowmeter connected with a valve; the splitter is configured such that a part of the 2-methyl-5-nitroimidazole sulfuric acid solution obtained after a first nitrification reaction in the second microreactor is used to replace the concentrated sulfuric acid to participate in a second nitrification reaction in the second microreactor to obtain a primarily-concentrated 2-methyl-5-nitroimidazole sulfuric acid solution; the splitter is opened to deliver the primarily-concentrated 2-methyl-5-nitroimidazole sulfuric acid solution to participate in a next step reaction, or the primarily-concentrated 2-methyl-5-nitroimidazole sulfuric acid solution is used to participate in a third nitrification reaction in the second microreactor to obtain a secondarily-concentrated 2-methyl-5-nitroimidazole sulfuric acid solution, and so forth until a saturated 2-methyl-5-nitroimidazole sulfuric acid solution is obtained, which is used in a next step through the splitter.

7. The full continuous synthesis method of claim 1, wherein the ethylene oxide conveying system comprises a buffer tank provided with an ethylene oxide inlet pipe and an ethylene oxide outlet pipe; the ethylene oxide inlet pipe is arranged in the buffer tank at ½ height of the buffer tank, and the ethylene oxide outlet pipe is configured to extend form a bottom of the buffer tank to a position inside the buffer tank which is away from the bottom of the buffer tank at a distance of ⅕-¼ height of the buffer tank; the buffer tank is pressurized to 5 bar-10 bar with nitrogen after filled with ethylene oxide; the ethylene oxide conveying system further comprises an injection pump; and the injection pump is configured to quantitatively feed the ethylene oxide to be mixed with a 2-methyl-5-nitroimidazole sulfuric acid-formic acid mixture to prepare metronidazole.

8. The full continuous synthesis method of claim 1, wherein in step (d), a pH value of the residual metronidazole solution is monitored by an on-line pH meter, and is adjusted with aqueous ammonia or liquid ammonia; the residual metronidazole solution is pumped to an on-line filter through a plunger pump to obtain a first filtrate; and the first filtrate is returned to a first pH-adjusting tank until a pH value of the first filtrate is kept within 2-6; and
the first filtrate with the pH value of 2-6 is adjusted with aqueous ammonia or liquid ammonia in a second pH-adjusting tank, pumped to the on-line filter through the plunger pump to obtain a second filtrate, and the second filtrate is returned to the second pH-adjusting tank until a pH value of the second filtrate is kept within 8-12; a filter cake of the on-line filter is continuously scraped by using a rotary scraper to obtain the crude metronidazole; and the crude metronidazole is decolorized and crystallized to obtain pure metronidazole with the purity greater than 99.9%.
